(19) United States
(12) Patent Application Publication
(10) Pub. No.: US 2026/0335338 A1
(43) Pub. Date: Oct. 29, 2026

(54) NEUTROPHILIC AIRWAY INFLAMMATION TREATMENT

(71) Applicant: UNIVERSITY OF SHARJAH, SHARJAH (AE)

(72) Inventors: Rabih HALWANI, Sharjah (AE); Bariaa KHALIL, Sharjah (AE); Narjes Saheb SHARIF-ASKARI, Sharjah (AE)

(21) Appl. No.: 19/635,161

(22) Filed: Mar. 31, 2026

Related U.S. Application Data
(60) Provisional application No. 63/780,632, filed on Mar. 31, 2025.

(51) Int. Cl. A61K 31/64; A61K 31/573; A61P 11/06; A61P 29/00

(52) U.S. Cl. CPC A61K 31/64; A61K 31/573; A61P 11/06; A61P 29/00

(57) ABSTRACT

A method of treating neutrophilic airway inflammation in a subject in need thereof comprises administering to the subject a therapeutically effective amount of an NLRP3 inflammasome inhibitor. The NLRP3 inflammasome inhibitor is MCC950. The neutrophilic airway inflammation may be associated with severe steroid hyporesponsive asthma or Th2-low asthma. The method may further comprise administering a corticosteroid to the subject. Administration of the NLRP3 inflammasome inhibitor may reduce IL-1β levels in bronchoalveolar lavage fluid, reduce neutrophil infiltration in the airways, reduce IL-17 levels in bronchoalveolar lavage fluid, and enhance responsiveness to corticosteroid treatment by reducing expression of glucocorticoid receptor beta. A pharmaceutical composition for treating neutrophilic airway inflammation comprises a therapeutically effective amount of an NLRP3 inflammasome inhibitor and a pharmaceutically acceptable carrier, wherein the NLRP3 inflammasome inhibitor is MCC950.

### CROSS-REFERENCE TO RELATED APPLICATIONS

This application claims priority to U.S. Provisional Application No. 63/780,632, titled “NEUTROPHILIC AIRWAY INFLAMMATION TREATMENT”, filed 31 Mar. 2025, which is hereby incorporated by reference in its entirety.

### FIELD OF INVENTION

The present disclosure relates to methods and compositions for treating inflammatory airway diseases, and more particularly to the use of NLRP3 inflammasome inhibitors for treating neutrophilic airway inflammation associated with severe steroid hyporesponsive asthma.

### BACKGROUND

Background description includes information that may be useful in understanding the present invention. It is not an admission that any of the information provided herein is prior art or relevant to the presently claimed invention, or that any publication specifically or implicitly referenced is prior art.

Corticosteroids are the first line of treatment and modulator of lung inflammation in asthmatic patients. In allergic asthma, corticosteroids skew TH1/TH2 balance and boost TH2 responses. Corticosteroids also promote the production of anti-inflammatory cytokine IL-10 by regulatory T cells. However, in patients with severe steroid hyporesponsive asthma, the anti-inflammatory effects of steroids are reduced due to the type of lung inflammation present. For instance, the increase in TH17 cells increases glucocorticoid receptor (GR)-β levels which in turn inhibits GR signaling. Hence, the inflammatory profile of asthmatic patients is a key determinant of their steroid responsiveness status.

In this regard, the inflammatory profile of patients with severe steroid hyporesponsive asthma revealed the activation of the NLRP3 inflammasome pathway in immune cells such as macrophages and neutrophils. Rossios et al. revealed using sputum transcriptomics on the U-BIOPRED cohort that neutrophil accumulation is characterized by inflammasome-associated gene signatures including TLR-2, NOD2, NLRP3, caspase-1, caspase-4, caspase-5 and IL-1R family. Further, Simpson et al. showed that sputum macrophages of neutrophilic asthmatics exhibited elevated expressions of TLR-2, TLR-4, NLRP3, caspase-1, caspase-4, caspase-5, and IL-1β. Sputum neutrophils showed strong immunoreactivity for NLRP3 and caspase-1 via immunocytochemical analysis. Importantly, increased expression of the innate immune receptors particularly TLR-2 and TLR-4 was accompanied by an increase in IL-1β and IL-8 in the sputum of patients with neutrophilic asthma. In addition, the upregulation in NLRP3 inflammasome is linked to the downstream production of GM-CSF and CCL-20 via IL-1R1 signaling. Hence, the NLRP3 inflammasome pathway is crucial in inducing neutrophil infiltration to the airways. This was evident in a study by Kim et al. who demonstrated the elevation of NLRP3 and IL-1β expression in a mouse model of severe, steroid-resistant airway disease. The group showed that the expression of NLRP3 and IL-1β was positively correlated with neutrophil numbers in patients' sputum and negatively correlated with airflow obstruction and asthma in a cohort of stable asthmatics, the majority of which were on inhaled corticosteroid therapy. Hence, the NLRP3 inflammasome pathway stands to be an important target for developing effective therapy for patients with severe steroid hyporesponsive asthma.

Currently, apart from anti-TSLP therapy, which can address Th2 high (eosinophilic) and Th2 low (non-eosinophilic) asthma, all biologics available are designed to treat severe eosinophilic asthma and there are no effective treatments for Th2 low neutrophilic asthma. Given the key involvement of NLRP3 inflammasome pathway in the pathogenesis of severe steroid hyporesponsive asthma and its association with disease severity, targeting this pathway therapeutically could potentially ameliorate disease severity and enhance responsiveness to steroids. This approach will not only have a great clinical impact on severe asthmatic patients by controlling disease symptoms and lessening asthma-related limitations in their daily lives but will also reduce the socioeconomic burden of severe asthma-related costs worldwide.

Therefore, improved methods and compositions for treating neutrophilic airway inflammation are desired.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a method of treating neutrophilic airway inflammation in a subject in need thereof is provided. The method comprises administering to the subject a therapeutically effective amount of an NLRP3 inflammasome inhibitor. The NLRP3 inflammasome inhibitor is MCC950.

According to other aspects of the present disclosure, the method may include one or more of the following features. The neutrophilic airway inflammation may be associated with severe steroid hyporesponsive asthma. The neutrophilic airway inflammation may be associated with Th2-low asthma. The method may further comprise administering a corticosteroid to the subject. The corticosteroid may be dexamethasone. The NLRP3 inflammasome inhibitor and the corticosteroid may be administered concurrently. The NLRP3 inflammasome inhibitor may be administered prior to administration of the corticosteroid. The NLRP3 inflammasome inhibitor may be administered by a route selected from the group consisting of oral administration, intranasal administration, intraperitoneal administration, intravenous administration, and inhalation. The NLRP3 inflammasome inhibitor may be administered at a dose of about 1 mg/kg to about 20 mg/kg body weight of the subject. Administration of the NLRP3 inflammasome inhibitor may reduce IL-1β levels in bronchoalveolar lavage fluid of the subject. Administration of the NLRP3 inflammasome inhibitor may reduce neutrophil infiltration in the airways of the subject. Administration of the NLRP3 inflammasome inhibitor may reduce IL-17 levels in bronchoalveolar lavage fluid of the subject. Administration of the NLRP3 inflammasome inhibitor may enhance responsiveness to corticosteroid treatment in the subject by reducing expression of glucocorticoid receptor beta in the subject. The method may further comprise administering a corticosteroid to the subject after administration of the NLRP3 inflammasome inhibitor. The subject may have severe steroid hyporesponsive asthma.

According to another aspect of the present disclosure, a pharmaceutical composition for treating neutrophilic airway inflammation is provided. The pharmaceutical composition comprises a therapeutically effective amount of an NLRP3 inflammasome inhibitor and a pharmaceutically acceptable carrier. The NLRP3 inflammasome inhibitor is MCC950.

According to other aspects of the present disclosure, the pharmaceutical composition may include one or more of the following features. The pharmaceutical composition may further comprise a corticosteroid. The corticosteroid may be dexamethasone. The pharmaceutical composition may be formulated for a dosage form selected from the group consisting of a tablet, a capsule, a solution for injection, an inhalable formulation, and a nasal spray. The pharmaceutical composition may be formulated for inhalation delivery to the airways.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 depicts a schematic timeline of an experimental protocol for inducing neutrophilic airway inflammation in a murine model, showing dexamethasone and MCC950 treatment dosing and schedule, according to aspects of the present disclosure.

FIG. 2 depicts experimental data demonstrating that MCC950 downregulates the NLRP3 inflammasome pathway at the activation step by reducing the protein expression of NLRP3 inflammasome (A, B) and rescuing the cleavage of caspase-1 (A, C), and that unlike dexamethasone, MCC950 significantly reduced the release of IL-1β in the bronchoalveolar lavage fluid (D), according to aspects of the present disclosure.

FIG. 3 depicts the BALF levels of IL-17 (A), neutrophil percentage (B), inflammation score (C) and mucus secretion as determined by PAS stain (D) compared between HDM/LPS mice with and without Dex or MCC950, according to aspects of the present disclosure.

FIG. 4 depicts bar graphs showing the fold change in the gene expression of glucocorticoid receptor isoforms GRα (A) and GRβ (B) compared between HDM/LPS mice with and without Dex or MCC950, according to aspects of the present disclosure.

Reference is made to the accompanying figures throughout the specification, which form a part hereof, and which is shown by way of illustration specific embodiments by which the invention may be practiced. It is to be understood that other embodiments may be utilized and logical changes may be made without departing from the scope of the present invention. The following detailed description and examples, therefore, is not to be taken in a limiting sense, and the scope of the present invention is defined by the appended claims.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure provides methods and compositions for treating neutrophilic airway inflammation in a subject in need thereof. The methods may comprise administering to the subject a therapeutically effective amount of an NLRP3 inflammasome inhibitor. In some aspects, the NLRP3 inflammasome inhibitor is MCC950, which is a selective and potent inhibitor of the NLRP3 inflammasome pathway.

Neutrophilic airway inflammation may be associated with severe steroid hyporesponsive asthma. In patients with severe steroid hyporesponsive asthma, the anti-inflammatory effects of corticosteroids may be reduced due to the type of lung inflammation present. The inflammatory profile of asthmatic patients may be a determinant of steroid responsiveness status. For instance, increases in certain T helper cell populations, such as TH17 cells, may elevate glucocorticoid receptor beta (GRβ) levels, which in turn may inhibit glucocorticoid receptor signaling.

Neutrophilic airway inflammation may also be associated with Th2-low asthma. Th2-low asthma, also referred to as non-eosinophilic asthma, may be characterized by neutrophil-dominant airway inflammation rather than eosinophil-dominant inflammation. In some cases, biologics available for treating severe asthma may be designed to address eosinophilic asthma, and there may be limited effective treatments for Th2-low neutrophilic asthma.

The NLRP3 inflammasome pathway may contribute to disease pathogenesis through activation of immune cells such as macrophages and neutrophils. In patients with severe steroid hyporesponsive asthma, the inflammatory profile may reveal activation of the NLRP3 inflammasome pathway in these immune cells. Neutrophil accumulation may be characterized by inflammasome-associated gene signatures, and sputum macrophages of neutrophilic asthmatics may exhibit elevated expressions of various inflammasome components including NLRP3, caspase-1, and IL-1β. Sputum neutrophils may show immunoreactivity for NLRP3 and caspase-1. The NLRP3 inflammasome pathway may induce neutrophil infiltration to the airways, and the expression of NLRP3 and IL-1β may be positively correlated with neutrophil numbers in patient sputum.

Given the involvement of the NLRP3 inflammasome pathway in the pathogenesis of severe steroid hyporesponsive asthma and the association of the NLRP3 inflammasome pathway with disease severity, targeting the NLRP3 inflammasome pathway therapeutically may ameliorate disease severity and enhance responsiveness to steroids. Inhibiting the NLRP3 inflammasome pathway may ameliorate the severity of Th2-low neutrophilic asthma and enhance responsiveness to corticosteroids.

The NLRP3 inflammasome inhibitor may be administered to a subject by various routes of administration. In some aspects, the NLRP3 inflammasome inhibitor may be administered orally. In some aspects, the NLRP3 inflammasome inhibitor may be administered intranasally. In some aspects, the NLRP3 inflammasome inhibitor may be administered intraperitoneally. In some aspects, the NLRP3 inflammasome inhibitor may be administered intravenously. In some aspects, the NLRP3 inflammasome inhibitor may be administered by inhalation. The route of administration may be selected based on the condition being treated, the desired site of action, and the characteristics of the subject.

The timing of administration of the NLRP3 inflammasome inhibitor may vary depending on the treatment regimen. In some aspects, the NLRP3 inflammasome inhibitor may be administered prior to allergen challenge. For example, the NLRP3 inflammasome inhibitor may be administered about 1 hour before allergen challenge. In some cases, administration of the NLRP3 inflammasome inhibitor prior to allergen challenge may provide prophylactic inhibition of the NLRP3 inflammasome pathway before exposure to an inflammatory stimulus.

The NLRP3 inflammasome inhibitor may be administered in combination with one or more additional therapeutic agents. In some aspects, the additional therapeutic agents may include corticosteroids. The NLRP3 inflammasome inhibitor and the corticosteroid may be administered according to various timing regimens. In some aspects, the NLRP3 inflammasome inhibitor may be administered prior to administration of the corticosteroid. In some aspects, the NLRP3 inflammasome inhibitor may be administered concurrently with the corticosteroid. In some aspects, the NLRP3 inflammasome inhibitor may be administered subsequently to administration of the corticosteroid.

In some cases, administration of the NLRP3 inflammasome inhibitor prior to corticosteroid administration may enhance the responsiveness of the subject to the corticosteroid. The NLRP3 inflammasome inhibitor may reduce expression of glucocorticoid receptor beta (GRβ), which may in turn enhance glucocorticoid receptor signaling and improve responsiveness to corticosteroid treatment. In some aspects, the corticosteroid may be dexamethasone. The corticosteroid may be administered by the same route as the NLRP3 inflammasome inhibitor or by a different route.

The NLRP3 inflammasome inhibitor may be administered in combination with a corticosteroid. In some aspects, the corticosteroid may be dexamethasone. The NLRP3 inflammasome inhibitor and the corticosteroid may be administered according to various timing regimens. In some aspects, the NLRP3 inflammasome inhibitor and the corticosteroid may be co-administered. In some aspects, the NLRP3 inflammasome inhibitor may be administered prior to administration of the corticosteroid. In some aspects, the NLRP3 inflammasome inhibitor may be administered subsequently to administration of the corticosteroid.

Combination treatment of the NLRP3 inflammasome inhibitor and the corticosteroid may have a synergistic effect on attenuating airway inflammation in neutrophilic asthma. The NLRP3 inflammasome inhibitor may reduce the activation of the NLRP3 inflammasome pathway, while the corticosteroid may provide additional anti-inflammatory effects. In some cases, the NLRP3 inflammasome inhibitor may reduce IL-1β levels in bronchoalveolar lavage fluid, whereas the corticosteroid alone may not reduce IL-1β levels. The combination of the NLRP3 inflammasome inhibitor and the corticosteroid may provide complementary mechanisms of action for treating neutrophilic airway inflammation.

Combination treatment may enhance responsiveness to corticosteroids in subjects with neutrophilic airway inflammation. In subjects with severe steroid hyporesponsive asthma, the anti-inflammatory effects of corticosteroids may be reduced due to elevated expression of glucocorticoid receptor beta (GRβ). The NLRP3 inflammasome inhibitor may reduce expression of GRβ, which may in turn enhance glucocorticoid receptor signaling. By reducing GRβ expression, the NLRP3 inflammasome inhibitor may restore or enhance the responsiveness of the subject to corticosteroid treatment.

The NLRP3 inflammasome pathway may be implicated in steroid resistance, and blocking the NLRP3 inflammasome pathway may enhance sensitivity to glucocorticoids. In some cases, administration of the NLRP3 inflammasome inhibitor prior to corticosteroid administration may enhance the therapeutic effect of the corticosteroid. The combination treatment may ameliorate disease severity in subjects with Th2-low neutrophilic asthma and may enhance responsiveness to corticosteroids in subjects who are otherwise hyporesponsive to corticosteroid treatment alone.

The NLRP3 inflammasome inhibitor may be administered at a therapeutically effective dose. In some aspects, the NLRP3 inflammasome inhibitor may be administered at a dose of about 1 mg/kg to about 20 mg/kg body weight of the subject. In some aspects, the NLRP3 inflammasome inhibitor may be administered at a dose of about 5 mg/kg to about 15 mg/kg body weight of the subject. In some aspects, the NLRP3 inflammasome inhibitor may be administered at a dose of about 8 mg/kg to about 12 mg/kg body weight of the subject. In some aspects, MCC950 may be administered at a dose of 10 mg/kg body weight of the subject.

The dose of the NLRP3 inflammasome inhibitor may be selected based on factors including the severity of the condition being treated, the route of administration, the age and weight of the subject, and the presence of other therapeutic agents in the treatment regimen. In some aspects, the NLRP3 inflammasome inhibitor may be administered at a dose of about 1 mg/kg body weight of the subject. In some aspects, the NLRP3 inflammasome inhibitor may be administered at a dose of about 2 mg/kg body weight of the subject. In some aspects, the NLRP3 inflammasome inhibitor may be administered at a dose of about 5 mg/kg body weight of the subject. In some aspects, the NLRP3 inflammasome inhibitor may be administered at a dose of about 15 mg/kg body weight of the subject. In some aspects, the NLRP3 inflammasome inhibitor may be administered at a dose of about 20 mg/kg body weight of the subject.

In some cases, the corticosteroid may be administered at a therapeutically effective dose. In some aspects, dexamethasone may be administered at a dose of about 0.5 mg/kg to about 5 mg/kg body weight of the subject. In some aspects, dexamethasone may be administered at a dose of about 0.5 mg/kg to about 2 mg/kg body weight of the subject. In some aspects, dexamethasone may be administered at a dose of 1 mg/kg body weight of the subject. The dose of dexamethasone may be selected based on factors including the severity of the condition being treated, the route of administration, and the responsiveness of the subject to corticosteroid treatment.

When the NLRP3 inflammasome inhibitor and the corticosteroid are administered in combination, the doses of each therapeutic agent may be adjusted based on the treatment regimen. In some aspects, MCC950 may be administered at a dose of 10 mg/kg body weight and dexamethasone may be administered at a dose of 1 mg/kg body weight. In some aspects, the NLRP3 inflammasome inhibitor may be administered at a lower dose when administered in combination with a corticosteroid compared to when administered alone. In some aspects, the corticosteroid may be administered at a lower dose when administered in combination with the NLRP3 inflammasome inhibitor compared to when administered alone.

Administration of the NLRP3 inflammasome inhibitor may produce various therapeutic effects in subjects with neutrophilic airway inflammation. In some aspects, administration of the NLRP3 inflammasome inhibitor may reduce IL-1β levels in bronchoalveolar lavage fluid (BALF) of the subject. The NLRP3 inflammasome inhibitor may downregulate the NLRP3 inflammasome pathway at the activation step, which may result in reduced release of IL-1β. In some cases, dexamethasone alone may not reduce IL-1β levels in subjects with neutrophilic airway inflammation, whereas the NLRP3 inflammasome inhibitor may reduce IL-1β release.

The NLRP3 inflammasome inhibitor may reduce the protein expression of NLRP3 inflammasome components. In some aspects, the NLRP3 inflammasome inhibitor may rescue the cleavage of caspase-1. The reduction in caspase-1 activation may contribute to the reduced release of IL-1β. The NLRP3 inflammasome pathway and IL-1β may be determinants of neutrophil infiltration to the airways.

Administration of the NLRP3 inflammasome inhibitor may reduce neutrophil infiltration in the airways of the subject. In some aspects, dexamethasone may not affect neutrophil infiltration in the bronchoalveolar lavage, whereas the NLRP3 inflammasome inhibitor may reduce neutrophil infiltration. The persistence of neutrophils in the presence of dexamethasone treatment may indicate the contribution of the NLRP3 inflammasome pathway in controlling neutrophil recruitment to the airways.

Administration of the NLRP3 inflammasome inhibitor may reduce IL-17 levels in bronchoalveolar lavage fluid of the subject. In some aspects, both dexamethasone and the NLRP3 inflammasome inhibitor may reduce IL-17 levels. However, the NLRP3 inflammasome inhibitor may provide additional therapeutic effects beyond IL-17 reduction, including reduction of neutrophil infiltration.

Administration of the NLRP3 inflammasome inhibitor may reduce the inflammation score in the subject. The NLRP3 inflammasome inhibitor may decrease the narrowing of the bronchial lumen, peribronchial infiltration, perivascular infiltration, and parenchymal infiltration of inflammatory cells. In some aspects, the NLRP3 inflammasome inhibitor may reduce the inflammation score to a greater extent than dexamethasone alone.

Administration of the NLRP3 inflammasome inhibitor may reduce mucus secretion in the airways of the subject. In some aspects, the NLRP3 inflammasome inhibitor may decrease the number of periodic acid-Schiff (PAS) positive cells, which are indicative of mucus-secreting cells. Both dexamethasone and the NLRP3 inflammasome inhibitor may reduce the number of PAS positive cells.

Administration of the NLRP3 inflammasome inhibitor may reduce total airway resistance in the subject. In some aspects, the NLRP3 inflammasome inhibitor may reduce total airway resistance and large airway resistance. In some cases, dexamethasone may not reduce total airway resistance in subjects with neutrophilic airway inflammation, whereas the NLRP3 inflammasome inhibitor may reduce airway resistance.

Administration of the NLRP3 inflammasome inhibitor may enhance responsiveness to corticosteroid treatment in the subject by reducing expression of glucocorticoid receptor beta (GRβ) in the subject. In subjects with neutrophilic airway inflammation, the expression of the inhibitory isoform GRβ may be elevated, which may contribute to reduced sensitivity to glucocorticoids. The NLRP3 inflammasome inhibitor may reduce the gene expression of GRβ.

The reduction in GRβ expression by the NLRP3 inflammasome inhibitor may indicate that the NLRP3 inflammasome pathway is implicated in steroid resistance. Blocking the NLRP3 inflammasome pathway may enhance sensitivity to glucocorticoids. In some aspects, subjects with neutrophilic airway inflammation may exhibit a dysregulation in the GRα/GRβ ratio, with decreased GRα expression and increased GRβ expression. The NLRP3 inflammasome inhibitor may reduce the expression of the inhibitory GRβ isoform, which may restore or enhance responsiveness to corticosteroid treatment.

A pharmaceutical composition for treating neutrophilic airway inflammation may comprise a therapeutically effective amount of an NLRP3 inflammasome inhibitor and a pharmaceutically acceptable carrier. In some aspects, the NLRP3 inflammasome inhibitor is MCC950. MCC950 is a selective and potent inhibitor of the NLRP3 inflammasome pathway. The pharmaceutical composition may be formulated for administration to a subject in need of treatment for neutrophilic airway inflammation, including neutrophilic airway inflammation associated with severe steroid hyporesponsive asthma or Th2-low asthma.

The pharmaceutically acceptable carrier may include one or more excipients suitable for the intended route of administration. In some aspects, the pharmaceutically acceptable carrier may include diluents, binders, lubricants, disintegrants, fillers, surfactants, solubilizers, stabilizers, preservatives, buffering agents, or combinations thereof. The selection of the pharmaceutically acceptable carrier may depend on the dosage form and the route of administration.

The pharmaceutical composition may further comprise a corticosteroid. In some aspects, the corticosteroid may be dexamethasone. The combination of the NLRP3 inflammasome inhibitor and the corticosteroid in a single pharmaceutical composition may provide complementary mechanisms of action for treating neutrophilic airway inflammation. In some cases, the NLRP3 inflammasome inhibitor may enhance responsiveness to the corticosteroid by reducing expression of glucocorticoid receptor beta (GRβ).

The pharmaceutical composition may be formulated for various dosage forms. In some aspects, the pharmaceutical composition may be formulated as a tablet. In some aspects, the pharmaceutical composition may be formulated as a capsule. Tablets and capsules may be suitable for oral administration of the NLRP3 inflammasome inhibitor. Tablets may include immediate-release tablets, extended-release tablets, or enteric-coated tablets. Capsules may include hard gelatin capsules, soft gelatin capsules, or capsules containing sustained-release formulations.

The pharmaceutical composition may be formulated as a solution for injection. In some aspects, the solution for injection may be formulated for intraperitoneal administration. In some aspects, the solution for injection may be formulated for intravenous administration. The solution for injection may include the NLRP3 inflammasome inhibitor dissolved or suspended in a sterile aqueous vehicle. The solution for injection may further include buffering agents, tonicity adjusting agents, preservatives, or combinations thereof.

The pharmaceutical composition may be formulated as an inhalable formulation. Inhalable formulations may be suitable for delivering the NLRP3 inflammasome inhibitor directly to the airways. In some aspects, the inhalable formulation may be a dry powder formulation for use with a dry powder inhaler. In some aspects, the inhalable formulation may be a solution or suspension for use with a nebulizer. In some aspects, the inhalable formulation may be a metered-dose inhaler formulation comprising the NLRP3 inflammasome inhibitor and a propellant.

The pharmaceutical composition may be formulated as a nasal spray. Nasal spray formulations may deliver the NLRP3 inflammasome inhibitor to the nasal passages and upper airways. In some aspects, the nasal spray may be an aqueous solution or suspension of the NLRP3 inflammasome inhibitor. The nasal spray may further include preservatives, buffering agents, tonicity adjusting agents, viscosity modifying agents, or combinations thereof.

The pharmaceutical composition may be formulated for inhalation delivery to the airways. Formulations for inhalation delivery may provide localized delivery of the NLRP3 inflammasome inhibitor to the site of neutrophilic airway inflammation. In some aspects, inhalation delivery may reduce systemic exposure to the NLRP3 inflammasome inhibitor while providing therapeutic concentrations at the airways. Inhalation formulations may include dry powder formulations, nebulizer solutions, nebulizer suspensions, or metered-dose inhaler formulations.

In some aspects, the pharmaceutical composition formulated for inhalation delivery may comprise particles of the NLRP3 inflammasome inhibitor having a particle size suitable for deposition in the lower airways. In some cases, the particles may have a mass median aerodynamic diameter of about 1 micrometer to about 5 micrometers. The particle size may be selected to provide deposition in the bronchi and bronchioles where neutrophilic inflammation may occur.

When the pharmaceutical composition comprises both the NLRP3 inflammasome inhibitor and a corticosteroid, the pharmaceutical composition may be formulated as a combination product. In some aspects, the combination product may be formulated as a fixed-dose combination tablet or capsule. In some aspects, the combination product may be formulated as a combination inhalable formulation. The combination inhalable formulation may deliver both the NLRP3 inflammasome inhibitor and the corticosteroid to the airways in a single administration.

The pharmaceutical composition may comprise the NLRP3 inflammasome inhibitor at a concentration suitable for achieving a therapeutically effective dose. In some aspects, the pharmaceutical composition may be formulated to deliver a dose of about 1 mg/kg to about 20 mg/kg body weight of the subject. In some aspects, the pharmaceutical composition may be formulated to deliver a dose of about 10 mg/kg body weight of the subject. When the pharmaceutical composition comprises dexamethasone, the dexamethasone may be present at a concentration suitable for delivering a dose of about 0.5 mg/kg to about 5 mg/kg body weight of the subject.

### EXAMPLES

The following examples are provided to illustrate aspects of the present disclosure and are not intended to limit the scope of the claims.

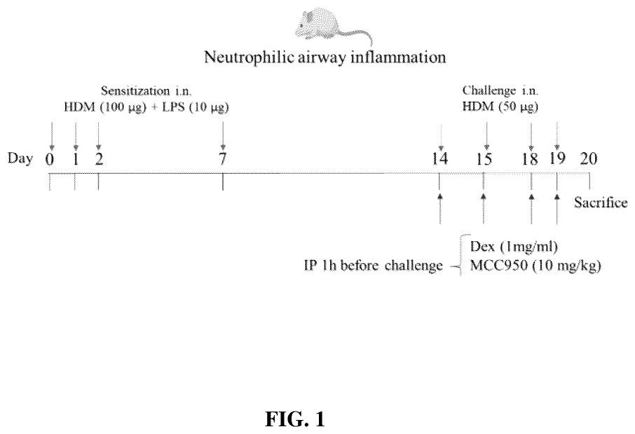

FIG. 1 depicts a schematic timeline of an experimental protocol for inducing neutrophilic airway inflammation in a murine model, showing dexamethasone and MCC950 treatment dosing and schedule, according to aspects of the present disclosure. A murine neutrophilic airway inflammation model was used to evaluate the efficacy of MCC950 in treating neutrophilic airway inflammation. The murine model served as a model for severe steroid hyporesponsive asthma. Wild type female BALB/cJ mice 8-10 weeks old weighing 18 g to 20 g were used as subjects. The mice were housed under pathogen-free and ovalbumin (OVA)-free conditions and kept in sterile, individually ventilated cages. The mice were maintained on a 12-hour light-dark cycle and fed a sterile maintenance diet and sterile-distilled water.

Mice experiments were performed following approval from the Animal Care and Use Committee at the University of Sharjah (agreement number: ACUC-20-02-11-01), as per the national guidelines for the care of laboratory animals.

With continued reference to FIG. 1, the murine neutrophilic airway inflammation model comprised a sensitization phase and a challenge phase. During the sensitization phase, house dust mite (HDM) extract and lipopolysaccharide (LPS) were administered intranasally to induce neutrophilic airway inflammation. The HDM extract was Dermatophagoides pteronyssinus extract. During the sensitization phase, 10 μg of LPS and 100 μg of HDM were administered intranasally to anesthetized mice on days 0, 1, 2, and 7. The mice were anesthetized with 5% isoflurane prior to intranasal administration. Environmental enrichment was provided by autoclaved dust-free aspen bedding.

As further shown in FIG. 1, the challenge phase followed the sensitization phase. During the challenge phase, HDM was administered intranasally to the mice. 50 μg of HDM was administered intranasally on days 14, 15, 18, and 19. The challenge phase induced an inflammatory response in the airways of the sensitized mice.

With continued reference to FIG. 1, treatment with the NLRP3 inflammasome inhibitor or a corticosteroid was administered during the challenge phase. MCC950 was administered at a dose of 10 mg/kg body weight via intraperitoneal injection. Dexamethasone was administered at a dose of 1 mg/kg body weight via intraperitoneal injection. The treatment was administered 1 hour before each HDM challenge during the challenge phase. Control mice received phosphate-buffered saline (PBS) during the sensitization and challenge phases.

Referring to FIG. 1, the mice were sacrificed on day 20 following completion of the challenge phase. Lung tissues, bronchoalveolar lavage fluid (BALF), and blood were collected from the mice for analysis. The collected samples were used to evaluate the effects of MCC950 treatment on the NLRP3 inflammasome pathway, airway inflammation, neutrophil infiltration, and glucocorticoid receptor expression.

After measurements of airway hyperresponsiveness (AHR), lungs were injected with ice-cold PBS-EDTA to collect the bronchoalveolar lavage cells. The following antibodies were used to characterize different immune cell populations: APC/Cyanine 7 anti-mouse CD45 (30-F11) (Cat. No. 103116), APC anti-mouse CD170 (Siglec F) (S17007L) (Cat. No. 155508), PercP-Cy5.5 anti-mouse CD11c (N418 ) (Cat. No. 117328), Alexa Fluor® 488 anti-mouse Ly-6G (1A8) (Cat. No. 127626), PE anti-mouse/human CD11b (M1/70) (Cat. No. 101208). All antibodies were purchased from Biolegend. The acquisition was performed on cytoFLEX flow cytometer (Beckman Coulter) using the cytoFLEX acquisition and analysis software version 2.3.0.84. Data was analyzed using FlowJo software (TreeStar Inc, Ashland, Ore) version 10.

When indicated, lungs were collected for histology and stored in 10% formalin. Lungs were embedded in paraffin blocks and cut into 5 μm sections and stained with hematoxylin and eosin (H&E) or periodic acid-Schiff (PAS, ab150680) according to the manufacturer's protocol. To score the level of perivascular and peribronchial inflammation, 6 representative fields/slide were evaluated and counted in a semi-quantitative blinded manner. A four point grading system was adopted for the following features: 0: no evidence of peribronchial, perivascular, and parenchymal infiltration of inflammatory cells, 1: few peribronchial, perivascular, and parenchymal infiltration of inflammatory cells, 2: one layer of peribronchial, perivascular, and parenchymal infiltration of inflammatory cells, 3: 2-4 layers of inflammatory cells in peribronchial, perivascular and parenchymal areas, 4: extensive infiltration of inflammatory cells (>4 layers of cell rings) in the peribronchial, perivascular, and parenchymal areas. For PAS staining, the scoring system depends on the quantification of the percentage of PAS-positive mucus-containing cells. A four grading system was used as follows: 0: no PAS positive cells, 1: <5% PAS positive cells, 2: 5-10% PAS positive cells, 3: 10-25% PAS positive cells and 4: >25% PAS positive cells.

Total RNA from mice lung tissue homogenates was extracted using Trizol (Invitrogen) method according to manufacturer instructions. RNA concentrations and purity were measured, followed by conversion to cDNA using High-Capacity cDNA Reverse Transcription Kit (Applied Biosystems). qRT-PCR reactions were performed using 5× Hot FirePol EvaGreen qRT-PCR SuperMix (Solis Biodyne) on QuantStudio 3 Real-Time PCR System (Applied Biosystems). Gene expression was analyzed using the fold change calculated by the equation 2^{-ΔΔCt} (ΔCT: Ct gene−Ct housekeeping & ΔΔCT: delta Ct exp.−delta Ct control) after normalization to the housekeeping gene EEF2. All results were expressed as mRNA expression (fold change) relative to unstimulated controls.

Proteins were obtained by homogenizing the lung tissue using RIPA buffer, supplemented with 1× Protease Inhibitor Cocktail (Sigma-Aldrich, Germany) and 1 mM phenylmethylsulfonyl fluoride (Sigma-Aldrich, Germany) followed by centrifugation at 14000 g for 20 min at 4 °C. Protein concentrations were measured using the BCA protein assay reagent kit (Thermo-Scientific Pierce BCA Protein Assay Kit). 20-40 μg total proteins were separated using 8%, 10% or 12.5% gels based on the different proteins' molecular weights. The proteins were transferred using semi-dry transfer cell onto a nitrocellulose membrane (Bio-Rad), blocked in skimmed milk for 1 h at room temperature, and incubated overnight at 4 °C with the various antibodies. Then, membranes were probed for 1 h with horseradish peroxidase-conjugated secondary antibodies. The blots were developed using the Clarity Western ECL Substrate (Bio-Rad) in the ChemiDoc Touch Gel Imaging System (Bio-Rad). Image J software was used to detect and quantify the protein bands.

The levels of IL-1β in mouse lung tissue lysates were determined using commercially available mouse IL-1Beta/IL-1F2 DuoSet ELISA kit (DY401-05). The levels of IL-17 in the BALF were quantified using IL-17A Mouse SimpleStep ELISA kit (Abcam, ab199081). Assays were performed following the manufacturer's instructions. All samples were measured in duplicates.

All data are presented as mean ±standard error of the mean (SEM) of 3-9 independent experiments. Statistical analysis was performed using unpaired two-tailed student's t-test, one-way ANOVA followed by S̆ídák's test for multiple comparisons, and two-way ANOVA with repeated measures followed by Tukey's multiple comparison test among different groups. A p value<0.05 was considered statistically significant. The statistical analysis was conducted using R software (version 3.0.2) and GraphPad Prism software, version 9.4.1 (GraphPad Software, Inc. La Jolla, CA, USA).

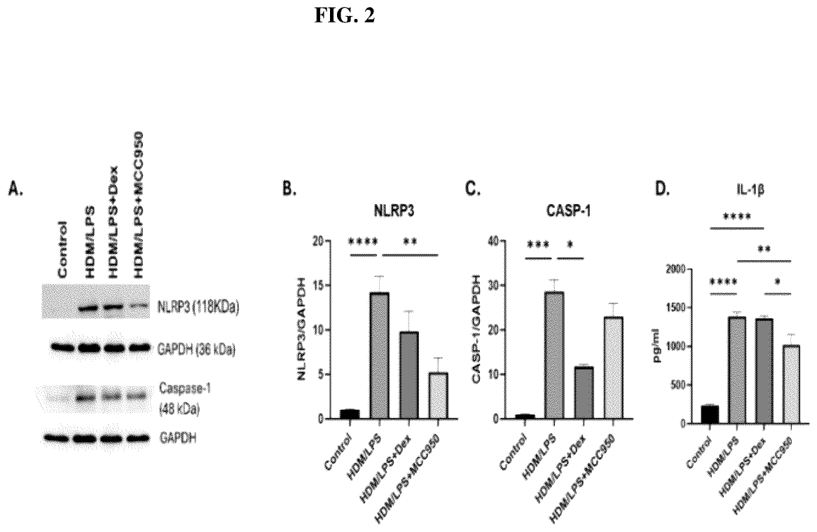

FIG. 2 depicts experimental data demonstrating that MCC950 downregulates the NLRP3 inflammasome pathway at the activation step by reducing the protein expression of NLRP3 inflammasome (A, B) and rescuing the cleavage of caspase-1 (A, C), and that unlike dexamethasone, MCC950 significantly reduced the release of IL-1β in the bronchoalveolar lavage fluid (D), according to aspects of the present disclosure. The effect of MCC950 on the NLRP3 inflammasome pathway was evaluated in the HDM/LPS mouse model. Western blot analysis was performed to assess the protein expression of NLRP3 inflammasome components in lung tissue lysates from control mice, HDM/LPS mice, HDM/LPS mice treated with dexamethasone, and HDM/LPS mice treated with MCC950. As shown in FIG. 2, Panel A, protein bands for NLRP3 (118 kDa), caspase-1 (36 kDa), and GAPDH (48 kDa) were detected across the four experimental groups.

With continued reference to FIG. 2, Panel B depicts quantification of NLRP3 protein expression normalized to GAPDH. HDM/LPS treatment increased NLRP3 expression compared to control mice. Both dexamethasone treatment and MCC950 treatment reduced NLRP3 protein levels in HDM/LPS mice. MCC950 was capable of downregulating the NLRP3 inflammasome pathway at the activation step, as detected by the reduction in the protein expression of NLRP3.

As further shown in FIG. 2, Panel C depicts quantification of caspase-1 (CASP-1) expression normalized to GAPDH. HDM/LPS mice exhibited elevated caspase-1 levels compared to control mice. MCC950 treatment rescued the cleavage of caspase-1 in HDM/LPS mice. Dexamethasone treatment induced a reduction in caspase-1 protein levels. The reduction in caspase-1 activation by MCC950 may contribute to the reduced release of IL-1β.

Referring to FIG. 2, Panel D depicts IL-1β concentration measurements in bronchoalveolar lavage fluid (BALF) across the experimental groups. Control mice exhibited minimal IL-1β levels in BALF. HDM/LPS mice exhibited markedly elevated IL-1β levels approaching 2000 pg/ml. MCC950 treatment produced a statistically significant reduction in IL-1β release in the BALF of HDM/LPS mice. Dexamethasone treatment showed less pronounced reduction in IL-1β levels compared to MCC950 treatment.

With continued reference to FIG. 2, MCC950 reduced the activation of caspase-1 and the consequent release of IL-1β, whereas dexamethasone failed to downregulate the activation of the NLRP3 inflammasome pathway. The persistent elevated levels of IL-1β in the presence of dexamethasone treatment indicated that dexamethasone did not reduce the activation of the NLRP3 inflammasome pathway. MCC950 was able to reduce the activation but not the priming of the NLRP3 inflammasome pathway. The NLRP3 inflammasome pathway and IL-1β may be determinants of neutrophil infiltration to the airways.

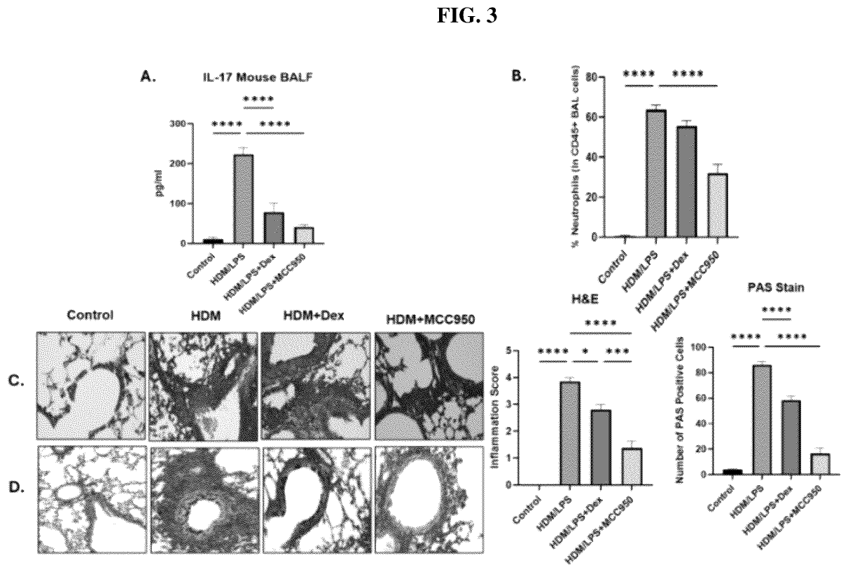

FIG. 3 depicts the BALF levels of IL-17 (A), neutrophil percentage (B), inflammation score (C) and mucus secretion as determined by PAS stain (D) compared between HDM/LPS mice with and without Dex or MCC950, according to aspects of the present disclosure. The effect of MCC950 on IL-17 levels, neutrophil infiltration to the bronchoalveolar lavage fluid (BALF), airway structural changes, and mucus secretion was evaluated in the HDM/LPS mouse model and compared against dexamethasone treatment. As shown in FIG. 3, Panel A, the levels of IL-17 in the BALF of asthmatic mice treated with dexamethasone were significantly reduced (P<0.0001). The levels of IL-17 in the BALF of asthmatic mice treated with MCC950 were also significantly reduced (P<0.0001).

With continued reference to FIG. 3, Panel B depicts the percentage of neutrophils among CD45-positive bronchoalveolar lavage cells. Dexamethasone did not affect neutrophil infiltration in the bronchoalveolar lavage as assessed by flow cytometry. In contrast, a significant decrease (P<0.0001) in neutrophil infiltration was detected in asthmatic mice treated with MCC950. The persistence of neutrophils in the presence of dexamethasone treatment may indicate the contribution of the NLRP3 inflammasome pathway in controlling neutrophil recruitment to the airways. The NLRP3 inflammasome pathway and IL-1β may be determinants of neutrophil infiltration to the airways.

As further shown in FIG. 3, Panels C and D depict histological sections of lung tissue stained with hematoxylin and eosin (H&E) and periodic acid-Schiff (PAS), respectively. Airway inflammation was evaluated by histological examination of lung tissue sections. Treatment with dexamethasone (P<0.05) and MCC950 (P<0.0001) decreased significantly the narrowing of the bronchial lumen, peribronchial infiltration, perivascular infiltration, and parenchymal infiltration of inflammatory cells in comparison to HDM/LPS mice. The H&E stained sections from the HDM/LPS group exhibited pronounced peribronchial and perivascular inflammation characterized by dense cellular infiltrates. The MCC950-treated group showed reduced inflammatory cell infiltration around bronchial and vascular structures.

With continued reference to FIG. 3, assessment of mucus secretion showed a significant decrease in the number of PAS positive cells in the HDM/LPS model upon treatment with dexamethasone and MCC950 (P<0.0001). The PAS stained sections from the HDM/LPS group showed substantial mucus accumulation, with mucus-secreting cells appearing in the bronchial epithelium. Both dexamethasone-treated and MCC950-treated groups demonstrated reduced mucus secretion compared to the untreated HDM/LPS group. The reduction in PAS positive cells may indicate decreased mucus production in the airways following treatment with the NLRP3 inflammasome inhibitor.

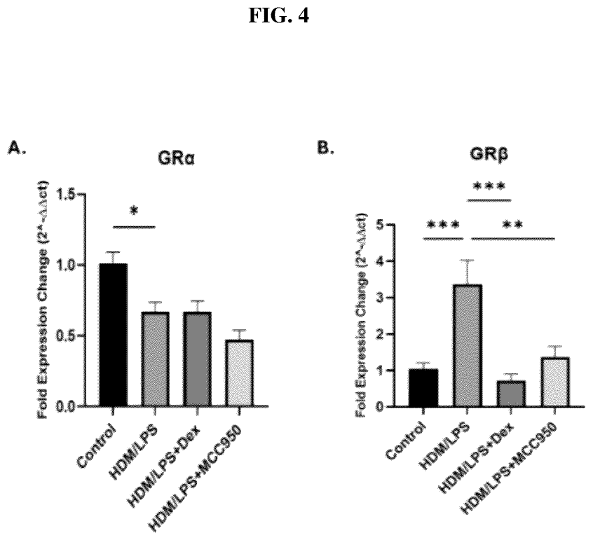

FIG. 4 depicts bar graphs showing the fold change in the gene expression of glucocorticoid receptor isoforms GRα (A) and GRβ (B) compared between HDM/LPS mice with and without Dex or MCC950, according to aspects of the present disclosure. The effect of MCC950 on steroid sensitivity was evaluated in the HDM/LPS mouse model. The mouse model was characterized to be hyporesponsive to steroids by determining the mRNA expression of glucocorticoid receptors GRα and GRβ. As shown in FIG. 4, Panel A, asthmatic mice exhibited a significant decrease in the expression of GRα (P<0.05) as compared with control mice. As shown in FIG. 4, Panel B, the decrease in GRα expression was associated with a significant increase in the gene expression of the inhibitory isoform GRβ (P<0.001). The HDM/LPS model induced a dysregulation in the GRα/GRβ ratio as an indication of hyporesponsiveness to glucocorticoids. Accordingly, the HDM/LPS mouse model was shown to be hyporesponsive to dexamethasone treatment, whereby dexamethasone failed to reduce total airway resistance at increasing concentrations of methacholine.

With continued reference to FIG. 4, the effect of treatment with dexamethasone and MCC950 on glucocorticoid receptor expression was evaluated. Treatment with dexamethasone and MCC950 did not increase the gene expression of GRα. As shown in FIG. 4, Panel B, a significant decrease in the gene expression of GRβ was detected in the presence of dexamethasone (P<0.001) and MCC950 (P<0.01). The dexamethasone-treated group exhibited reduced GRβ expression to approximately 0.7 fold change compared to control. The MCC950-treated group exhibited reduced GRβ expression to approximately 1.2 fold change compared to the HDM/LPS group, which exhibited approximately 3.5 fold change in GRβ expression.

As further shown in FIG. 4, although MCC950 did not show a significant improvement in the GRα/GRβ ratio, the significant reduction in the gene expression of GRβ indicates that the NLRP3 inflammasome pathway may be implicated in steroid resistance. Blocking the NLRP3 inflammasome pathway may enhance sensitivity to glucocorticoids. The reduction in GRβ expression by MCC950 may restore or enhance responsiveness to corticosteroid treatment in subjects with neutrophilic airway inflammation.

A number of implementations have been described. Nevertheless, it will be understood that various modifications may be made without departing from the spirit and scope of the disclosure. Accordingly, other implementations are within the scope of the following claims.

The treatment protocol may pave the way to clinical trials that may help ameliorate the severity of symptoms in patients with Th2-low (neutrophilic) asthma and reduce the associated socioeconomic burden by enhancing patients' responsiveness to corticosteroids.

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents. The disclosures and the description herein are intended to be illustrative and are not in any sense limiting the invention, defined in scope by the following claims. Many changes, modifications, variations and other uses and applications of the subject invention will become apparent to those skilled in the art after considering this specification and the accompanying drawings, which disclose the preferred embodiments thereof. All such changes, modifications, variations and other uses and applications, which do not depart from the spirit and scope of the invention, are deemed to be covered by the invention, which is to be limited only by the claims which follow.

1. A method of treating neutrophilic airway inflammation in a subject in need thereof, comprising:
administering to the subject a therapeutically effective amount of an NLRP3 inflammasome inhibitor,
wherein the NLRP3 inflammasome inhibitor is MCC950.

2. The method of claim 1, wherein the neutrophilic airway inflammation is associated with severe steroid hyporesponsive asthma.

3. The method of claim 1, wherein the neutrophilic airway inflammation is associated with Th2-low asthma.

4. The method of claim 1, further comprising administering a corticosteroid to the subject.

5. The method of claim 4, wherein the corticosteroid is dexamethasone.

6. The method of claim 4, wherein the NLRP3 inflammasome inhibitor and the corticosteroid are administered concurrently.

7. The method of claim 4, wherein the NLRP3 inflammasome inhibitor is administered prior to administration of the corticosteroid.

8. The method of claim 1, wherein the NLRP3 inflammasome inhibitor is administered by a route selected from the group consisting of oral administration, intranasal administration, intraperitoneal administration, intravenous administration, and inhalation.

9. The method of claim 1, wherein the NLRP3 inflammasome inhibitor is administered at a dose of about 1 mg/kg to about 20 mg/kg body weight of the subject.

10. The method of claim 1, wherein administration of the NLRP3 inflammasome inhibitor reduces IL-1β levels in bronchoalveolar lavage fluid of the subject.

11. The method of claim 1, wherein administration of the NLRP3 inflammasome inhibitor reduces neutrophil infiltration in the airways of the subject.

12. The method of claim 1, wherein administration of the NLRP3 inflammasome inhibitor reduces IL-17 levels in bronchoalveolar lavage fluid of the subject.

13. The method of claim 1, wherein administration of the NLRP3 inflammasome inhibitor enhances responsiveness to corticosteroid treatment in the subject by reducing expression of glucocorticoid receptor beta in the subject.

14. The method of claim 13, further comprising administering a corticosteroid to the subject after administration of the NLRP3 inflammasome inhibitor.

15. The method of claim 13, wherein the subject has severe steroid hyporesponsive asthma.

16. A pharmaceutical composition for treating neutrophilic airway inflammation, comprising:
a therapeutically effective amount of an NLRP3 inflammasome inhibitor; and
a pharmaceutically acceptable carrier,
wherein the NLRP3 inflammasome inhibitor is MCC950.

17. The pharmaceutical composition of claim 16, further comprising a corticosteroid.

18. The pharmaceutical composition of claim 17, wherein the corticosteroid is dexamethasone.

19. The pharmaceutical composition of claim 16, wherein the pharmaceutical composition is formulated for a dosage form selected from the group consisting of a tablet, a capsule, a solution for injection, an inhalable formulation, and a nasal spray.

20. The pharmaceutical composition of claim 16, wherein the pharmaceutical composition is formulated for inhalation delivery to the airways.
